(19) United States
(12) Patent Application Publication
(10) Pub. No.: US 2020/0281638 A1
(43) Pub. Date: Sep. 10, 2020

(54) CATHETER SYSTEM FOR CRYOABLATION, IN PARTICULAR CRYOABLATION OF THE GASTRIC WALL

(71) Applicant: Georg Daniel Andreas Nollert, Strasslach (DE)

(72) Inventor: Georg Daniel Andreas Nollert, Strasslach (DE)

(21) Appl. No.: 16/802,749

(22) Filed: Feb. 27, 2020

(30) Foreign Application Priority Data
Mar. 8, 2019 (DE) 10 2019 105 970.4

(51) Int. Cl. A61B 18/02

(52) U.S. Cl. CPC A61B 18/02; A61B 2018/0022

(57) ABSTRACT

A catheter system for cryoablation is described that comprises: a catheter, which extends along its longitudinal axis; at least one cryoballoon which surrounds the catheter around its entire circumference and forms a filling lumen; a cooling lumen, which is separate from the filling lumen, within the cryoballoon, wherein the cooling lumen is arranged at the outer region of the cryoballoon away from the longitudinal axis of the catheter; at least one filling conduit which extends within the catheter and terminates in the filling lumen; and at least one cooling conduit which extends within the catheter and terminates in the cooling lumen.

### CROSS-REFERENCE TO RELATED APPLICATION

This application claims the benefit of and priority to German Patent Application No. DE 10 2019 105 970.4, filed Mar. 8, 2019, the contents of which being incorporated by reference in their entirety herein.

### TECHNICAL FIELD

The present invention relates to a catheter system for cryoablation, in particular for reducing gastric motility in the case of obesity.

### BACKGROUND

Obesity is the cause of many lifestyle diseases such as diabetes, damage to joints, or heart failure. In addition to non-invasive treatments such as dieting or sporting activities, there is also the countermeasure of invasive treatments such as a sleeve gastrectomy, gastric band operation, intragastric balloon, gastric bypass, gastric pacemaker, injecting Botox or ablating the gastric branch of the vagus nerve.

The principle of circumferential cryoablation is applied when ablating pulmonary veins in atrial fibrillation. It has been shown that obesity can also be treated by (multiple) circumferential cryoablation of the gastric wall, wherein parts of the gastric muscles are frozen, such that restricted peristalsis occurs and food remains in the stomach for longer, thus suppressing the sense of hunger. Inhibiting peristalsis in the stomach also means that components of food, in particular fats, are less well dissolved, such that resorption of the food is reduced.

### BRIEF SUMMARY OF INVENTION

The present invention relates to a catheter system using which cryoablation, in particular cryoablation of the gastric wall, can be performed.

The catheter system comprises a catheter, which extends along its longitudinal axis, and at least one cryoballoon which surrounds the catheter around its entire circumference and forms a filling lumen. A catheter is usually an elongated flexible tube. The circumference of the catheter extends in a plane which is perpendicular to the longitudinal axis of the catheter, wherein the circumference denotes the line formed in this plane by the outermost points on the surface of the catheter.

A catheter has a distal end and a proximal end, wherein the distal end is intended to be inserted into a body through an opening. The at least one cryoballoon is arranged at the distal region of the catheter, such that it can be inserted into the body together with the distal end of the catheter. In the case of a catheter system for gastric cryoablation, the distal end of the catheter—together with the at least one cryoballoon—is for example inserted into the stomach through the esophagus. In the following, the term “inserting the catheter” denotes inserting the distal end of the catheter.

The catheter system also comprises a cooling lumen, which is separate from the filling lumen, within the cryoballoon, wherein the cooling lumen is arranged at the outer region of the cryoballoon away from the longitudinal axis of the catheter. The catheter system also comprises at least one filling conduit, which extends within the catheter and terminates in the filling lumen, and at least one cooling conduit which extends within the catheter and terminates in the cooling lumen.

A cryoballoon is thus a balloon which, when used, at least partially contains a coolant, in order to perform cryoablation. It comprises an outer shell which has an inner end, which faces the catheter, and an outer end which faces away from the inner end. The inner end is for example fastened to the catheter in order to prevent the cryoballoon from shifting along the catheter. The cooling lumen lies within the shell of the cryoballoon, in particular at the outer end of the shell. It is for example annular and surrounds the circumference of the catheter.

The cryoballoon has an outer diameter at its outer end and an inner diameter at its inner end. The term “outer region” means a region which begins at the outer end and extends towards the inner end. In the radial direction, the cooling lumen and therefore the outer region extends for example over up to 30%, preferably 20%, more preferably between 5% and 10% of the difference between the outer diameter and the inner diameter.

The at least one cryoballoon is deflated while the catheter is being inserted. In order for a cryoballoon to assume its desired shape and for example abut the inner side of the gastric wall, the filling lumen is filled with a suitable filler, for example air or a filling liquid such as for example a saline solution, through a filling conduit. Filling the filling lumen in particular causes the cryoballoon to circumferentially abut the surrounding tissue, for example the gastric wall. The cooling lumen is filled with a coolant, such as for example a cooling liquid, via the at least one cooling conduit. Since the cooling lumen is situated in the outer region of the cryoballoon, the coolant contained therein cools the surrounding tissue through the shell of the cryoballoon.

In one embodiment of the invention, the catheter system comprises a reservoir for the filler, the reservoir being connected to the filling conduit at the distal end of the catheter. The catheter system optionally comprises a conveying device by means of which the filler can be conveyed from the reservoir into the filling lumen and/or from the filling lumen into the reservoir.

In one embodiment, the catheter system comprises a pressure sensor which measures the pressure in the filling conduit as the filling lumen of the cryoballoon is filled. It is thus possible to output an indication and/or stop the conveying device as soon as a predetermined threshold value of the pressure in the filling conduit is reached or exceeded. Alternatively or additionally, the catheter system can comprise a pressure limiter which automatically limits the pressure in the filling conduit. It is thus possible to prevent overfilling of the cryoballoon.

In one embodiment of the invention, the catheter system comprises a reservoir for the coolant. This reservoir is connected to the at least one cooling conduit at the proximal end of the catheter. Optionally, the catheter system also comprises a conveying device which conveys the coolant from the reservoir into the cooling lumen via the cooling conduit.

Since a cryoballoon surrounds the catheter around its entire circumference, a cryoballoon has an outer circumference. In one embodiment of the invention, the cooling lumen extends over at least 90% of the outer circumference of the cryoballoon, preferably at least 95%, 98% or 99%. It is thus possible to simultaneously cool an entire circumferential strip of for example the gastric wall.

In one embodiment of the invention, a cryoballoon is rotationally symmetrical and for example toroidal. The cross-section of the torus is for example circular, ovate, oval or elliptical.

The rotational axis of the cryoballoon is for example the longitudinal axis of the catheter. It should be noted that the rotational symmetry of the cryoballoon relates at least to its outer shape, since the internal structure may be asymmetrical, for example due to a connection between the cooling lumen and the cooling conduit.

In one embodiment of the invention, the at least one cooling conduit comprises a feed conduit and a return conduit. In this embodiment, the cooling lumen is a conduit which is arranged in the outer region of the cryoballoon. One end of this conduit is connected to the feed conduit; the other end of this conduit is connected to the return conduit. The two ends of the conduit are preferably arranged immediately adjacent, such that the coolant flows through the filling lumen along the entire outer circumference of the cryoballoon. In this embodiment, new coolant can be continuously fed into the cooling lumen, thus providing a more constant cooling capacity for the catheter system.

In one embodiment of the invention, the catheter system comprises a pH sensor at the distal end of the catheter. This pH sensor measures the pH value and transmits it for example to a display device. The position of the distal end of the catheter can be ascertained from the measured pH value. The pH value in the stomach is for example lower than the pH value in the duodenum. The pH sensor is for example arranged in a soft flexible tube, made for example of silicone, which extends the catheter at its distal end.

In one embodiment of the invention, the catheter system comprises two or more cryoballoons which are arranged next to each other on the catheter. This enables multiple regions, for example regions of the gastric wall, to be ablated simultaneously.

It is then possible to provide each cryoballoon with its own filling conduit in the catheter or to provide a shared filling conduit for all the cryoballoons or to provide a multitude of filling conduits, each of which is assigned to a disjunct subset of the available cryoballoons. Equally, it is possible to provide each of the cryoballoons with its own cooling conduit or to provide a shared cooling conduit for all the cryoballoons or to provide a multitude of cooling conduits, each of which is assigned to a disjunct subset of the available cryoballoons. One or more or all of the cooling conduits can (respectively) comprise a feed conduit and a return conduit.

In one embodiment of the invention, the catheter system also comprises a distal balloon which is arranged on the catheter between the distal end of the catheter and the most distal cryoballoon, wherein the distal balloon has for example a smaller diameter than the cryoballoons. The lumen formed by the distal balloon is for example connected to a filling conduit or a cooling conduit. The distal balloon can thus also be deflated while the catheter is being inserted and for example filled only once it is in the stomach. The distal balloon can be placed in the duodenum, inflated and retracted as far as the pylorus. The distal balloon, and therefore the distal end of the catheter, can thus be anchored in the pylorus. If the distal balloon is filled with a coolant, then the pylorus can also be cooled.

In one embodiment of the invention, the catheter system also comprises a proximal balloon which is arranged proximally with respect to the most proximal cryoballoon. The proximal balloon is for example connected to a cooling conduit, such that it can be filled with a coolant. The cardia can be cooled by means of the proximal balloon.

The proximal balloon can be arranged on the catheter. Alternatively, the proximal balloon can be arranged on a second catheter which can be shifted in the longitudinal direction relative to the catheter. The catheter system can thus be adapted to different anatomies. The second catheter extends for example concentrically around the catheter.

In one embodiment of the invention, the catheter comprises a depth scale, by means of which the insertion depth of the catheter into a body can be ascertained. If the anatomy is known, the insertion depth of the catheter corresponds to the position or positions of the at least one cryoballoon in for example the stomach. In the present case, the insertion depth denotes the length of the portion of the catheter which is inserted into the body. Another option is to ascertain, for example by means of another catheter, the length of the trajectory between the entry point into the body and a point in the stomach such as the pylorus or the transition into the duodenum, in order to thence determine the required insertion depth of the catheter in accordance with the invention.

In one embodiment of the invention, the shell of the cryoballoon exhibits a greater wall thickness and/or another material in the region of the cooling lumen than outside this region and/or than a separation between the cooling lumen and the filling lumen. This prevents the coolant from exiting the catheter system, for example because the boundary of the filling lumen is damaged, thus deflating the cryoballoon, before the boundary of the cooling lumen is damaged. In another scenario, damage to the shell of the cooling lumen is more likely to occur in the direction of the filling lumen than towards the outside of the cryoballoon.

In the case of a catheter system for gastric cryoablation, the diameter of a cryoballoon is for example 20 to 30 cm, preferably 23 to 27 cm, more preferably 25 cm. The diameter of a distal balloon or proximal balloon is for example 3 to 8 cm, preferably 5 cm. The distance between two cryoballoons or between the distal balloon and the most distal cryoballoon is for example 2 to 5 cm.

### BRIEF DESCRIPTION OF THE DRAWINGS

The present invention shall be described in more detail on the basis of specific example embodiments. The corresponding figures, which are not true-to-scale, show:

FIG. 1 a frontal view of a catheter system;

FIG. 2 a lateral sectional representation of the catheter system from FIG. 1;

FIG. 3 a frontal sectional representation of the catheter system from FIG. 1;

FIG. 4 another catheter system, comprising a multitude of balloons; and

FIG. 5 a catheter comprising a depth scale.

### DETAILED DESCRIPTION

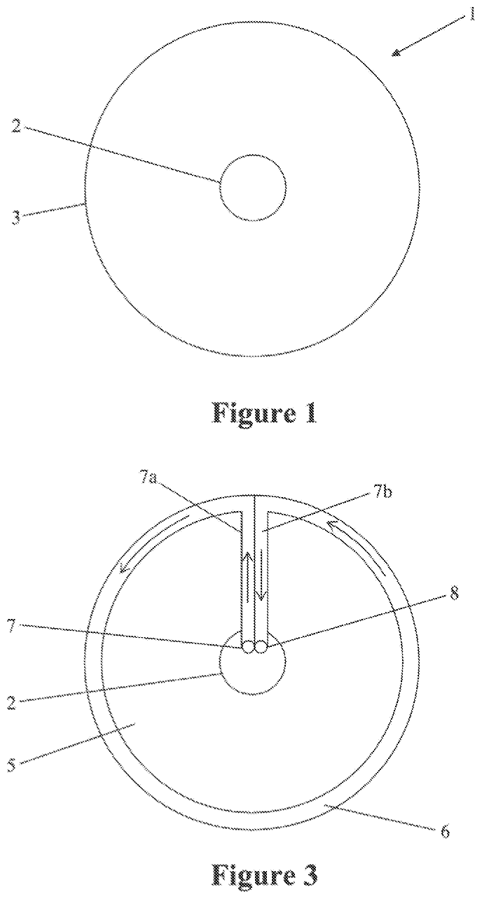

FIG. 1 shows a schematic frontal view of a catheter system 1 comprising a catheter 2 and a cryoballoon 3. The catheter 2 extends along its longitudinal axis, which in the representation in FIG. 1 extends into the plane of the paper. The view in FIG. 1 shows the tip of the catheter, i.e. the distal end of the catheter 2.

The cryoballoon 3 surrounds the catheter 2 around its entire circumference. The cryoballoon 3 has an inner circumference which abuts and is connected to the outer face of the catheter 2 over its entire circumference, such that the cryoballoon 3 is spatially fixed relative to the catheter 2 in the direction of the longitudinal axis of the catheter 2.

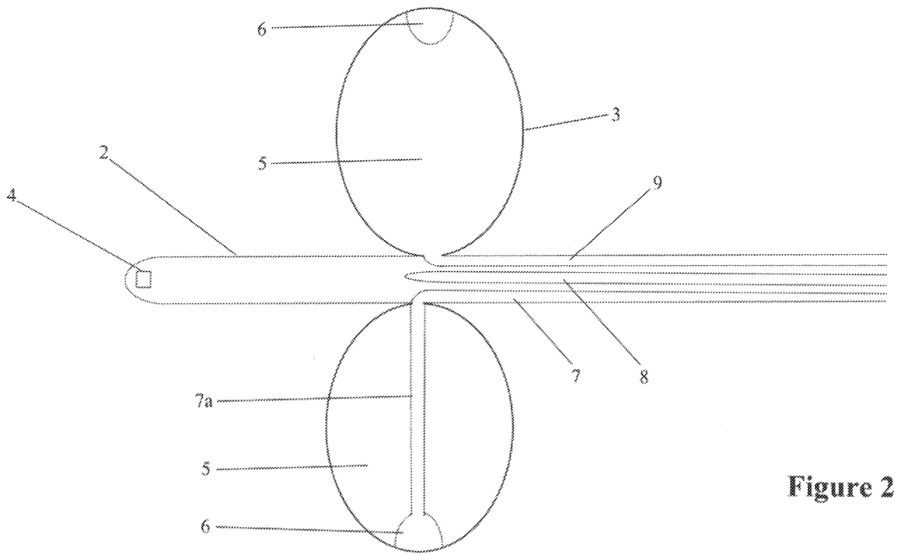

FIG. 2 shows a schematic lateral sectional representation through the catheter system 1 from FIG. 1, in which it can be seen that the cryoballoon 3 which surrounds the catheter 2 has, by way of example, an oval or elliptical cross-section. The outer shape of the cryoballoon 3 is rotationally symmetrical about the longitudinal axis of the catheter 2. The majority of the inner volume of the cryoballoon 3 is taken up by a filling lumen 5, although the cryoballoon 3 also contains a cooling lumen 6 which is situated at the outer periphery, in relation to its distance from the catheter 2, within the cryoballoon 3. The cooling lumen 6 is delineated from the filling lumen 5 by means of a boundary.

A filling conduit 9, which is connected to the filling lumen 5, extends within the catheter 2. A cooling conduit, which comprises a feed conduit 7 and a return conduit 8, also extends within the catheter 2. The feed conduit 7 is connected to the cooling lumen 6 via a connecting piece 7a which extends within the filling lumen 5. The return conduit 8 is likewise connected to the cooling lumen 6 via a connection 7b, not shown in FIG. 2, through the filling lumen 5. A coolant, for example a cooling liquid, is conveyed via the feed conduit 7 to an entry point in the cooling lumen 6 and passes through the cooling lumen 6 up to an exit point at which it is guided back via the connection 7b and the return conduit 8.

At its distal end, the catheter 2 also comprises a pH sensor 4 which measures the pH value of a substance at the distal end of the catheter 2, converts it into an electrical signal and transmits the electrical signal, through a conduit in the catheter 2 which is not shown in the figures, to a control device arranged outside the catheter 2.

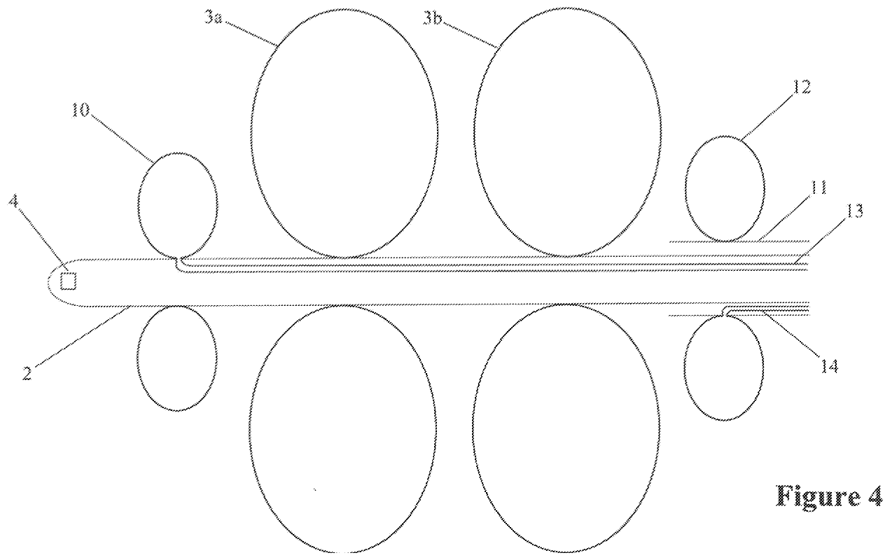

FIG. 3 schematically shows a frontal sectional representation through the catheter system 1 from the same direction of view as in FIG. 1. As is clear from FIG. 3, the cooling lumen 6 surrounds the entire circumference of the catheter 2 in a ring-like manner. FIG. 3 also shows the connection 7b which connects the exit point of the cooling lumen 6 to the return conduit 8.

The cryoballoon 3 consists of an elastic material. The boundary between the filling lumen 5 and the cooling lumen 6 within the cryoballoon 3 likewise consists of an elastic material.

Use of the catheter system 1 for cryoablation of the gastric wall is described in the following.

The distal end of the catheter 2 is inserted into the stomach through the esophagus, while the cryoballoon 3 is deflated. Once the catheter 2 is situated in the desired position, a filler such as for example a saline solution is conveyed into the filling lumen 5 via the filling conduit 9, until the outer circumference of the cryoballoon 3 abuts the gastric wall. A coolant is then fed via the feed conduit 7 and enters into the cooling lumen 6 at the entry point via the connection 7a, whence the coolant passes through the cooling lumen 6, as symbolized by the arrows in FIG. 3. The coolant thus cools a circumferential strip of the gastric wall. At the exit point of the cooling lumen 6, the coolant is conveyed back via the connection 7b and the return conduit 8. This results in a constant flow of coolant through the cooling lumen 6.

As can be seen from FIG. 3, the entry point and exit point of the cooling lumen 6 are immediately adjacent, such that the cooling lumen 6 extends over the entire outer circumference of the cryoballoon 3.

The term “outer circumference” in this document means the line which extends once around the entire circumference of the catheter 2 and which is formed by the points which lie on the surface of the cryoballoon 3 and are furthest away from the catheter 2 when the cryoballoon 3 is inflated, i.e. when the filling lumen 5 is filled.

FIG. 4 shows a schematic sectional representation through a modified catheter system 14 which by way of example comprises cryoballoons 3a and 3b arranged on the catheter 2, wherein the cryoballoon 3a is nearer to the distal end of the catheter 2 than the cryoballoon 3b. A distal balloon 10 is arranged between the distal end of the catheter 2 and the most distal cryoballoon 3a. The distal balloon 10 surrounds the catheter 2 around its entire circumference and has a smaller outer diameter than the cryoballoons 3a and 3b. The catheter 2 likewise comprises a pH sensor 4 at its distal end.

The catheter 2 is concentrically surrounded by a second catheter 11. The second catheter 11 surrounds the catheter 2 in a region which is proximal with respect to the most proximal cryoballoon 3b. A proximal balloon 12 is arranged on the second catheter 11 and surrounds the second catheter 11, and therefore the catheter 2, around its entire circumference. The proximal balloon 12 is thus arranged proximally with respect to the most proximal cryoballoon 3b. It has for example the same outer radius as the distal balloon 10.

The second catheter 11 can be shifted relative to the catheter 2 in the direction of the longitudinal axis of the catheter 2 and therefore in the direction of the longitudinal axis of the second catheter 11. The proximal balloon 12 can thus be shifted relative to the distal balloon 10 and the cryoballoons 3a and 3b.

A supply conduit 13 extends within the catheter 2 and is connected to the inner space of the distal balloon 10. A supply conduit 14 extends within the second catheter 11 and is connected to the inner space of the proximal balloon 12. The distal and/or proximal balloon can be filled with a filler and/or coolant via the supply conduits 13 and 14. The two balloons can also respectively be completely or partially evacuated again via the supply conduits.

When using the modified catheter system for cryoablation of the gastric wall, the distal end of the catheter 2 is inserted into the stomach while repeatedly ascertaining the pH value by means of the pH sensor 4. It is possible to ascertain, from a change in the pH value, when the distal end of the catheter 2 has reached the stomach. The catheter 2 is then inserted further, and from an increase in the pH value, it is ascertained that the distal end of the catheter 2 has reached the duodenum. The distal balloon 10 is then inflated, by feeding a coolant or a combination of a filler and a coolant via the supply conduit 13. The catheter 2 is then retracted until the distal balloon 10 is situated in the pylorus and cools it. All the cryoballoons 3a and 3b are then inflated, gradually or simultaneously, by filling the corresponding filling lumen 5 with a filler. The coolant is then conveyed into the cooling lumens 6 of the cryoballoons 3a and 3b in order to cool the gastric wall. The proximal balloon 12 is inflated, by feeding a coolant or a combination of a coolant and filler via the supply conduit 14. By means of shifting the second catheter 11 relative to the catheter 2, the proximal balloon 12 is guided up to the cardia and cools it.

Instead of separate supply conduits 13 and 14 for the distal balloon 10 and/or proximal balloon 12, it is also possible to provide a shared supply conduit which is connected to the inner volume of both the distal balloon 10 and the proximal balloon 12.

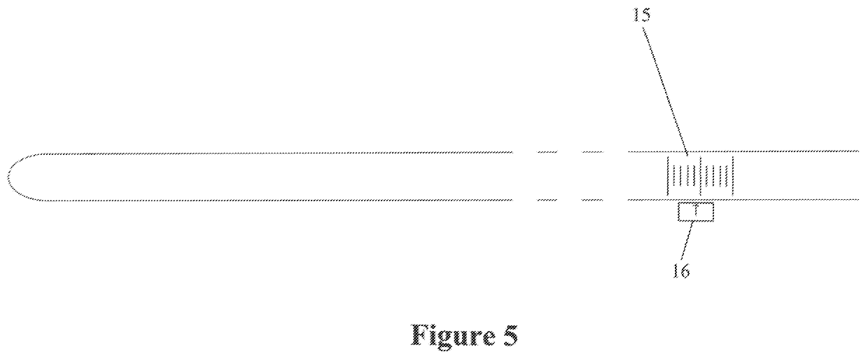

FIG. 5 schematically shows a catheter 2 comprising a depth scale arranged on it. When the catheter 2 is inserted, it moves along its longitudinal axis past a reference 16 which is for example spatially fixed relative to the entry point of the catheter 2. The insertion depth of the catheter 2 can be determined from the depth scale 15, from which the position of the distal end of the catheter 2 can for example be derived.

Therefore, the following is claimed:

1. A catheter system for cryoablation, comprising:
a catheter, which extends along its longitudinal axis;
at least one cryoballoon which surrounds the catheter around its entire circumference and forms a filling lumen;
a cooling lumen, which is separate from the filling lumen, within the cryoballoon, wherein the cooling lumen is arranged at an outer region of the cryoballoon away from the longitudinal axis of the catheter;
at least one filling conduit which extends within the catheter and terminates in the filling lumen; and
at least one cooling conduit which extends within the catheter and terminates in the cooling lumen.

2. The catheter system according to claim 1, wherein the cooling lumen extends over at least 90 percent of an outer circumference of the cryoballoon.

3. The catheter system according to claim 1, wherein the cryoballoon is rotationally symmetrical.

4. The catheter system according to claim 1, wherein the at least one cooling conduit comprises a feed conduit and a return conduit.

5. The catheter system according to claim 1, further comprising a pH sensor at a distal end of the catheter.

6. The catheter system according to claim 1, further comprising two or more cryoballoons which are arranged next to each other on the catheter.

7. The catheter system according to claim 6, further comprising a distal balloon arranged on the catheter between the distal end of the catheter and a most distal one of the cryoballoons.

8. The catheter system according to claim 7, further comprising a proximal balloon which is arranged proximally with respect to a most proximal one of the cryoballoons.

9. The catheter system according to claim 8, wherein the proximal balloon is arranged on a second catheter, the proximal balloon configured to be shifted in the direction of the longitudinal axis of the catheter relative to the catheter.

10. The catheter system according to claim 1, wherein the catheter comprises a depth scale through which an insertion depth of the catheter into a body can be ascertained.
